(19) United States
(12) Patent Application Publication
(10) Pub. No.: US 2010/0273260 A1
(43) Pub. Date: Oct. 28, 2010

(54) CELL CULTURE SUBSTRATE AND PROCESS FOR PRODUCING THE SAME AND METHOD FOR CULTURING CELLS

(75) Inventors: Chiharu Odane, Ibaraki-shi (JP); Sakura Toshikawa, Ibaraki-shi (JP); Atsuko Mizuike, Ibaraki-shi (JP); Hiroyuki Nishii, Ibaraki-shi (JP); Takuji Shintani, Ibaraki-shi (JP); Carsten Werner, Dresden (DE); Mirko Nitschke, Dresden (DE); Walter Eevers, Genk (BE)

Correspondence Address: SUGHRUE-265550, 2100 PENNSYLVANIA AVE. NW, WASHINGTON, DC, 20037-3213, US

(73) Assignee: NITTO DENKO CORPORATION, Ibaraki-shi (JP)

(21) Appl. No.: 12/600,037

(22) PCT Filed: Jul. 10, 2008
(86) PCT No.: PCT/JP2008/062869

§ 371 Date: Nov. 13, 2009

(30) Foreign Application Priority Data
Jul. 11, 2007 (JP) 2007-181913

(51) Int. Cl. C12N 5/02; C12M 1/00; C08G 65/00; C08G 63/08

(52) U.S. Cl. 435/396; 435/289.1; 528/392; 528/354

(57) ABSTRACT

The present invention relates to a cell culture substrate in which a polymer chain having a hydrophilic skeleton is grafted onto a surface of polystyrene or poly(ε-caprolactone) having a water contact angle of from 75° to 100°. This cell culture substrate has excellent efficiency of cell culture without the necessity of immobilization and adsorption of a cell adhesion substance on a surface of a substrate.

### TECHNICAL FIELD

The present invention relates to a cell culture substrate which is for use in culturing cells. More specifically, it relates to a cell culture substrate which can grow cells safely and efficiently without the need of immobilization and adsorption of a cell adhesion substance.

### BACKGROUND ART

In recent years, regeneration repair of defective tissues via regeneration induction of biotissues has been enabled. However, the existing surgical therapy completely dependent on biomaterials and artificial organs has drawbacks that the therapeutic effect thereof is temporal with great invasion and functions which can be supplemented are simple. Meanwhile, organ transplantation suffers from problems such as lack of organ donors and a rejection reaction after transplantation.

Under these circumstances, regeneration medical treatment has attracted much interest as a new therapy. The basic concept of regeneration medical treatment is that functions of defective tissues and damaged organs are regeneratively repaired by promoting growth and differentiation of cells using cells or tissues artificially cultured to induce regeneration of self-tissues.

In general, when cells are cultured in vitro, it is important that cells are cultured at high density upon maintaining the function of cells at the same level as in vivo. In order to grow cells by adhering them to a culture substrate, it is required that adhesion of the cells to the substrate surface is good and the adhered cells are in a state capable of extension, growth and migration.

The cell culture has been so far performed using a glass, a culture dish made of polymer, a test tube, a culture bottle or the like. A high-molecular material, which has been hitherto used as a cell culture substrate, especially polystyrene is excellent in moldability, durability, transparency, nontoxicity and low cost. However, since the surface of polystyrene is hydrophobic, it is not appropriate in view of cellular adhesiveness. Further, polystyrene has suffered from a problem that on the hydrophobic surface of polystyrene, uncontrollable interaction occurs between the cells and the adsorptive protein on the surface to cause adsorption and denaturation of the protein on the surface.

Accordingly, a cell culture substrate, in which the hydrophobic surface of polystyrene is subjected to corona discharge treatment to introduce an anion into the surface alone to impart hydrophilicity and improve the adhesiveness and growth of cells, has been developed and widely used. However, it has been found that the corona discharge treatment makes it hard to develop specific functions of cells and maintain the same for a long period of time.

As an approach to inhibit adsorption of a protein which decreases efficiency of cell culture by hydrophilizing a hydrophobic surface of a cell culture substrate, there are methods in which a hydrophilic compound such as a collagen gel or a hydrophilic polymer is coated on the surface (see, Patent Document 1). Nevertheless, in these methods, there has been a possibility that the hydrophilic compound coated on the surface is eluted in a medium aqueous solution during the cell culture thereby to cause an adverse effect on performance and morphology of cells.

On the other hand, there is a method in which hydrophilicity is imparted to the surface by immobilizing a hydrophilic compound on the hydrophobic surface of the cell culture substrate. It is reported that according to this method, a high-density hydrous layer is formed by the hydrophilic compound extending from the surface into a medium aqueous solution and an exclusion volume effect is increased, whereby the adsorption of a protein is inhibited. In this method, however, it has been confirmed that when cells are cultured on the surface, the cellular adhesiveness is low (see, Patent Document 2).

Thus, a technique in which functions such as adhesion, growth, differentiation and substance production of cells are improved by making an environment of cell culture as close to an in-vivo state as possible has been actively developed. For example, a technique using an extracellular matrix and a cell adhesion substance such as a cell adhesion factor has been developed (see, Patent Document 3).

The extracellular matrix refers to a synthetic biopolymer which is synthesized in cells and excreted and accumulated extracellularly. The extracellular matrix is a structural support of a tissue precipitated around the cells, and it is a substance that controls cell adhesion, orientation of a cell skeleton, a cell form, cell migration, cell growth, intracellular metabolism and cell differentiation. Examples of the extracellular matrix include collagen as a main component, and fibronectin, laminin, vitronectin, proteoglycan and glycosaminoglycan as a second component.

The cell adhesion factor refers to a factor which is present on a cell surface and is involved in adhesion of an intracellular or extracellular matrix. Examples of the factor which is involved in intracellular adhesion include cadherin family, Ig super family, selectin family and sialumtin family. Examples of the factor which is involved in adhesion of an extracellular matrix include integrin family.

Examples of a culture substrate on which an extracellular matrix component for controlling adhesion and growth of cells is immobilized include a cell culture substrate coated with collagen (see, Non-patent Document 1), a cell culture substrate coated with fibronectin (see, Non-patent Document 2), and a cell culture substrate coated with a cell adhesion protein (see, Non-patent Document 3).

However, it is not easy to reproduce in vitro a higher-order structure of an extracellular matrix immobilized on the surface of the cell culture substrate. It has been very difficult to realize it with good reproducibility. Further, there has been generally a problem that the cell adhesion substance is quite costly.

Patent Document 1: JP-A-6-153905

Patent Document 2: JP-T-2002-511496 (the term JP-T as used herein means a published Japanese translation of a PCT patent application)

Patent Document 3: JP-A-2004-208692

Non-patent Document 1: K. Yoshizato et al. Annals of Plastic Surgery. Vol. 13, No. 1 1984

Non-patent Document 2: F. Grinnell. Expl. Cell Res., 102. 51. 1984

Non-patent Document 3: P. T. Piccioano. et al., In Vitro Cellular and Developmental Biology 22(3). 24A. 1986

### DISCLOSURE OF THE INVENTION

The invention aims to provide, upon solving such ordinary problems, a cell culture substrate having excellent efficiency of cell culture without the need of immobilization and adsorption of a cell adhesion substance on a substrate surface.

The present inventors have assiduously conducted investigations in consideration of the foregoing problems, and have consequently found that, according to a cell culture substrate in which a polymer chain having a hydrophilic skeleton is grafted onto a surface of polystyrene or poly(ε-caprolactone) having a water contact angle of from 75° to 100°, efficiency of cell culture can be improved without the necessity of immobilization or adhesion of a cell adhesion substance. The invention has been completed based on this finding. That is, the invention relates to the following items 1 to 8.

1. A cell culture substrate in which a polymer chain having a hydrophilic skeleton is grafted onto a surface of polystyrene or poly(ε-caprolactone) having a water contact angle of from 75° to 100°.
2. The cell culture substrate according to item 1, wherein the surface of the polystyrene or poly(ε-caprolactone) grafted with the polymer chain having a hydrophilic skeleton has a water contact angle of from 25° to 60°.
3. The cell culture substrate according to item 1 or 2, wherein the polymer chain having a hydrophilic skeleton is at least one member selected from the group consisting of polyethylene oxide, polypropylene oxide, and derivatives or copolymers thereof.
4. The cell culture substrate according to any one of items 1 to 3, wherein the polymer chain having a hydrophilic skeleton has a molecular weight of from 500 to 2,000.
5. A method for culturing cells, which comprises using the cell culture substrate according to any one of items 1 to 4.
6. A process for producing a cell culture substrate, comprising steps of:

(1) forming a surface of polystyrene or poly(s-caprolactone) having a water contact angle of from 75° to 100°, and

(2) grafting a polymer chain having a hydrophilic skeleton onto the surface of the polystyrene or poly(ε-caprolactone) formed in the step (1).

7. The process according to item 6, wherein the grafting of the polymer chain having a hydrophilic skeleton in the step (2) is conducted in accordance with plasma graft copolymerization.
8. The process according to item 6 or 7, wherein a water contact angle of the surface of the polystyrene or poly(ε-caprolactone) grafted with the polymer chain having a hydrophilic skeleton in the step (2) is 25° to 60°.

The cell culture substrate of the invention makes it possible to improve efficiency of the cell culture without the necessity of immobilization or adsorption of the cell adhesion substance on the substrate surface.

### BRIEF DESCRIPTION OF THE DRAWINGS

FIG. 1 shows results of evaluation of adsorption of a protein.

FIG. 2 shows results of evaluation of efficiency of cell culture.

### BEST MODE FOR CARRYING OUT THE INVENTION

The invention is described in detail below.

The invention relates to a cell culture substrate in which a polymer chain having a hydrophilic skeleton is grafted to a hydrophobic polymer skeleton.

The hydrophobic polymer used in the invention is a polymer having a water contact angle of from 75° to 100°. Herein, the water contact angle means a value which can be calculated by the following formula when a liquid droplet is put on a surface of a solid and equilibrium is reached in this atmosphere. The following formula is called “Young's formula”, and an angle which is formed by a liquid surface and a solid surface is defined as a “contact angle”. The water contact angle can be measured by a commercially available apparatus, for example, DropMaster 500 (manufactured by Kaimen Kagaku).

γₛ=γ_{L} cos Θ+γ_{SL}

(wherein γₛ represents a surface tension of a solid, γ_{L}, represents a surface tension of a liquid, γ_{SL}, represents a solid/liquid surface tension, and Θ represents a contact angle.)

The hydrophobic polymer may be either biodegradable or non-biodegradable depending on a desired application. Further, it is preferable that the hydrophobic polymer is nontoxic, biocompatible and excellent in mechanical stability, transparency and moldability so as to be useful either in vivo or in vitro.

Examples of the hydrophobic polymer include engineering plastics such as polystyrene, polyethylene, polypropylene, polyester, polycarbonate, polyamide and polyacetal, and general-purpose polymers, and mixtures thereof.

As the hydrophobic polymer, biodegradable polymers may be also used. Examples thereof include poly(ε-caprolactone), polylactic acid, polyhydroxybutyric acid, polyethylene adipate and polybutylene carbonate. These biodegradable polymers are preferable in view of their in-vivo absorption when they are used in medical treatment.

Among the hydrophobic polymers, polystyrene is preferable since the transparency, processability and strength thereof are excellent. Further, in view of easy procurement, cost and the like, poly(ε-caprolactone) and polylactic acid are preferable. Moreover, in view of solubility in an organic solvent, polybutylene carbonate and polyethylene carbonate are preferable.

Examples of the polymers having a hydrophilic skeleton as used in the invention include polymers containing a hydroxyl group such as polyethylene glycol, polyethylene oxide, polypropylene glycol, polyvinyl alcohol, polyhydroxyethyl methacrylate and polypropylene oxide; polymers containing a vinyl group such as polyvinyl pyrrolidone; polymers containing an acid amide group such as polyacrylamide and polymethacrylamide; and copolymers with monomers constituting the same. Among these, polyethylene oxide, polypropylene oxide, and derivatives and copolymers thereof are preferable in view of the resistance properties to sterilization using radial ray. These may be used solely or two or more kinds thereof may be used in combination.

It is preferable that the molecular weight of the polymer chain having a hydrophilic skeleton is from 500 to 2,000. This is because it is possible to maintain the orientation of the polymer chain well when the molecular weight of the polymer chain is within this range. When the polymer chain having a hydrophilic skeleton is a copolymer, the composition ratio thereof is not particularly limited, and the rate of the hydrophilic group contained in the molecule can be freely controlled by varying the composition ratio. It is preferable that the polymer chain having a hydrophilic skeleton is grafted in an amount of 5 part by weight or more, more preferably 10 parts by weight or more, based on 100 parts by weight of the hydrophobic polymer skeleton. When the amount thereof is 5 parts by weight or more, it is possible to obtain sufficient hydrophilicity and it also becomes possible to graft the polymer chain having a hydrophilic skeleton uniformly.

A method of grafting the polymer chain having a hydrophilic skeleton on the hydrophobic polymer skeleton is not particularly limited. Examples of the graft copolymerization include plasma graft copolymerization, photograft copolymerization and radiation graft copolymerization. Among these, plasma graft copolymerization is preferable, since introduction of a polar group such as an amino group, an amide group, a carboxyl group, a carbonyl group, an ester group or a hydroxyl group into the surface and formation of a chemical bond are enabled by selecting a type of a process gas used in a gas plasma treatment and wide-ranging usage is possible (see, for example, B. J. Jeong et al., J. Colloid Interf. Sci., 178, 757 (1996).).

These methods are generally used as a polymer surface modification method in which a solid phase-type reaction is conducted. According to these methods, a polymer radical is formed on whole the molecular chain of the polymer by to a high energy source, and formation of the polymer radical is conducted only on the surface layer. Thus, these methods are most appropriate for modification of the surface layer.

Specific examples of the methods by which a polymer chain having a hydrophilic skeleton is grafted to a hydrophobic polymer skeleton in accordance with graft copolymerization include the following methods (A) to (C).

(A) A polymer radical is previously formed on whole the molecular chain of a hydrophobic polymer by a high energy source to form a reactive functional group. Polymerization is then conducted in an aqueous solution of a polymer chain having a hydrophilic skeleton with a dehydrating agent or a reaction initiator (see, for example, Z. Cheng, S-H. Tech, Biomaterials 25 (2004) 1991-2001.). In this case, it is necessary that an end or one or more side chains of the polymer chain having a hydrophilic skeleton be capable of reacting with the reactive functional group formed in the hydrophobic polymer skeleton. Examples of the reactive functional group include a carboxyl group, a hydroxyl group, and an amino group.

(B) A polymer radical is previously formed on whole the molecular chain of a hydrophobic polymer by a high energy source, followed by being contacted with a reaction solution of a polymer chain having a hydrophilic skeleton which has a double bond to conduct a grafting (see, for example, Nakayama Y et. al., ASAIO, 39, M754-M757 (1993).). In this case, it is necessary that an end or one or more side chains of the polymer chain having the hydrophilic skeleton have a double bond. Examples of such a polymer chain having a hydrophilic skeleton include poly(N,N-dimethylacryl amid) and polymethacryloyloxy alkylphosphoryl choline.

(C) A monomer forming a hydrophilic polymer skeleton is introduced into or contacted with a reaction system to conduct a radical polymerization via a radical reaction initiator (see, for example, Nakayama Y et. al., Macromolecules, 32, 5405-5410 (1999).). In this case, it is necessary that the monomer or macromonomer having a hydrophilic skeleton have a polymerizable chain end. Examples of the radical reaction initiator include α,α-azobisisobutyronitrile (AIBN) and benzoyl peroxide (BPO). Examples of the monomer or macromonomer having a polymerizable chain end include N-vinylacetamid, methyl acrylate, and methyl methacrylate.

According to the invention, after a polymer chain having a hydrophilic skeleton is grafted onto a surface of the hydrophobic polymer, the surface of the hydrophobic polymer preferably has a water contact angle of from 25° to 60°.

The cell culture substrate of the invention may have any form, and it may have a flat surface, a curved surface, a cylindrical surface or the like. Among these, it is preferably a flat surface in the step of grafting the polymer chain having a hydrophilic skeleton.

The form of the substrate having a flat surface is provided by, for example, subjecting a base material to hot-melt pressing. Alternatively, for example, a hydrophobic polymer is dissolved in an appropriate solvent, and the solution is subjected to a spin coating, dipping or casting on an inorganic material substrate such as a glass, a metal or a silicon wafer, or a substrate of polymer excellent in resistance to organic solvent, such as polypropylene, polyethylene or polyether ketone thereby forming the substrate having a flat surface. In view of surface smoothness, the inorganic material substrate is preferably used.

The types of the cells which can be cultured on the cell culture substrate according to the invention are not particularly limited. Animal cells, especially adhesive cells may be mentioned. The cell culture substrate can widely be used in culturing, for example, fibroblasts, smooth muscle cells, vascular endothelial cells, corneal cells, cartilaginous cells, hepatic cells, small intestinal epithelial cells, epidermal keratinocytes, osteoblasts, bone marrow mesenchymal cells, germinal stem cells, adult stem cells, nerve stem cells and neurons, regardless of strain cells or primary cells. Further, it can be also used in culturing so-called floating cells such as blood cells.

When the cell culture substrate of the invention is used, efficiency of the cell culture can be improved without the necessity of immobilization and adsorption of a cell adhesion substance such as an extracellular matrix or a cell adhesion factor. The cell culture substrate of the invention can be applied to substrates used so far in the cell culture, such as a petri dish, a plate, an incubator, a culture bag, a film, a fiber, a microcarrier and beads, or other substrates which can be used in the cell culture.

A specific example of the process for producing the cell culture substrate according to the invention is a process comprising the following steps (1) to (2).

(1) Step of Forming a Surface of a Hydrophobic Polymer

Step (1) is a step of forming a surface of a hydrophobic polymer. The hydrophobic polymer is dissolved in a solvent such as chloroform or tetrahydrofuran in an amount of from 0.3 to 1.0% by mass to prepare a hydrophobic polymer aqueous solution. The hydrophobic polymer aqueous solution is spin-coated on a polymer substrate, and allowed to stand still and dried at room temperature to form a hydrophobic polymer-containing thin film having a film thickness of from 30 to 150 nm. The spin coating conditions satisfy a maximum rotational number of from 3,000 to 5,000 rpm and a period of from 30 to 60 seconds.

(2) Grafting of a Polymer Chain Having a Hydrophilic Skeleton onto the Surface of the Hydrophobic Polymer

Step (2) is a step of grafting a polymer chain having a hydrophilic skeleton onto the surface of the hydrophobic polymer skeleton formed in step (1). As stated above, the grafting method is not particularly limited. However, grafting using the method (A) in accordance with plasma graft copolymerization is described herein.

The hydrophobic polymer-containing thin film prepared in step (1) is allowed to stand still in a reactor of a low-pressure plasma generator (for example, Plasma Beam manufactured by Diner Electronic Corporation, or Micro Systems Apparatus manufactured by Roth & Rau AG), and an atmospheric pressure inside the reactor is adjusted to from 1×10⁻³ to 1×10⁻² mbar using a vacuum pump. As a gas in the reactor, nitrogen, argon, ammonia, water, hydrogen, sulfur oxide and the like are used, and a gas flow rate is from 10 to 100 cm³/sec. Subsequently, the hydrophobic polymer-containing thin film is treated with an output of 100 to 500 W for 10 to 600 seconds using a plasma generation source (for example, Electron Cyclotron Resonance manufactured by Roth & Rau AG, or radiofrequency wave generator manufactured by Roth & Rau AG) to generate plasma in the reactor. At this time, a distance between the thin film and the plasma generation source is approximately 100 to 1,000 mm.

The polymer chain having the hydrophilic skeleton is dissolved in a liquid compound such as water or phosphate buffer solution (PBS) in a proportion of from 5 to 20 mM to prepare a polymer chain aqueous solution having a hydrophilic skeleton, and pH is set at 7.0 to 7.5. In the polymer aqueous solution, a condensing agent such as diisopropylcarboziimide or 1-ethyl-3-(3-dimethylaminopropyl)carboziimide hydrochloride may be dissolved, and a concentration of the condensing agent in the polymer aqueous solution may be set at 50 to 100 mM. In the polymer aqueous solution, the hydrophobic polymer-containing thin film treated with plasma in step (2) is dipped, and then allowed to stand still at room temperature for 12 to 24 hours. Thereafter, the thin film is rinsed with distilled water, and allowed to stand still and dried at room temperature.

### EXAMPLES

### Examples 1 and 2

### Preparation of a Polystyrene Surface

In Examples 1 and 2, polystyrene represented by the following formula (1) was used as a hydrophobic polymer. Polystyrene (manufactured by TOYO STYRENE: molecular weight approximately 4,300,000) was dissolved in chloroform (purchased from WAKO CHEMICAL) at a concentration of 0.6% w/w to prepare a hydrophobic polymer solution. The hydrophobic polymer solution was spin-coated on a silicon wafer (for measuring a contact angle) of 10×20 mm² or a cover glass (for cell culture) having a diameter of 15 mm to form a thin film having a film thickness of approximately 65 nm. The spin coating conditions were that a maximum rotational number was 3,000 rpm and a time was 30 seconds. After the spin coating, the product was allowed to stand still and dried at room temperature.

In a sample for protein adsorption test, an ultrathin film of less than 60 nm has to be formed on a metallic thin film sensor chip (measurable lower limit is 60 nm). Accordingly, polystyrene was dissolved in chloroform to adjust a concentration to 0.3% w/w, and the solution was spin-coated on the surface to form a thin film having a film thickness of approximately 30 nm. The spin coating conditions were that a maximum rotational number was 3,000 rpm and a time was 30 seconds. After the spin coating, the product was allowed to stand still and dried at room temperature.

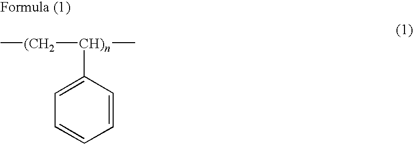

(Grafting of a Polymer Chain Having a Hydrophilic Skeleton on a Surface of a Polystyrene)

A polystyrene-containing thin film formed on a slide glass was allowed to stand still in a reactor of a low-pressure plasma generator, and an atmospheric pressure in the reactor was adjusted to 5×10⁻³ mbar using a vacuum pump. An oxygen gas was used as a gas in the reactor, and a gas flow rate was 10 cm³/sec. Plasma was then generated in the reactor with an output of 250 W using a 2.46 GHz electron cyclotron resonance-type magnetic field microwave generation source. At this time, a distance between the sample and the plasma generation source was approximately 200 mm. The plasma treatment of the sample was conducted for 30 seconds.

Subsequently, a polymer chain having a hydrophilic skeleton was grafted on the oxygen plasma-treated polystyrene-containing thin film. In Example 1, a copolymer of polypropylene oxide (hereinafter also referred to as PO) and polyethylene oxide (hereinafter also referred to as EO) having an amino group in one end [trade name: Jeffamine XTJ-506, molecular weight: approximately 1,000, copolymerization ratio (mol ratio) PO:EO=3:19, manufactured by HUNTSMAN], which is represented by the following formula (2), was used as a polymer chain having a hydrophilic skeleton. Then, 10 mM of this polymer and 50 mM of 1-ethyl-3-(3-diaminopropylcarbodiimide) as a condensing agent were mixed at 1/15 M with phosphate buffered saline to prepare an aqueous solution having pH of 7.4.

In Example 2, a copolymer of polypropylene oxide (PO) and polyethylene oxide (EO) having an amino group in one end [trade name: Jeffamine, molecular weight: approximately 2,000, copolymerization ratio (mol ratio) PO:EO=10:31, manufactured by HUNTSMAN], which is represented by the following formula 2, was used as a polymer chain having a hydrophilic skeleton.

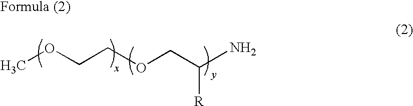

In the above formula (2), R is H in the case of EO, and R is CH₃ in the case of PO.

The polystyrene-containing thin film treated with oxygen plasma in the foregoing method was taken out of the reactor, immediately contacted with the polymer chain aqueous solution having a hydrophilic skeleton, and allowed to stand still at room temperature for 1 hour. Thereafter, the surface was rinsed with distilled water, and then allowed to stand still and dried at room temperature.

### Example 3

### Preparation of a Surface of a poly-ε-caprolactone

In Example 3, poly-ε-caprolactone represented by the following formula 3 was used as a hydrophobic polymer. Poly-ε-caprolactone (purchased from WAKO CHEMICAL: weight average molecular weight from 70,000 to 100,000) was dissolved in chloroform (purchased from WAKO CHEMICAL) at a concentration of 1.25% w/w to prepare a hydrophobic polymer aqueous solution. The hydrophobic polymer aqueous solution was spin-coated on a silicon wafer (for measuring a contact angle) of 10×20 mm² or a cover glass (for cell culture) having a diameter of 15 mm to form a poly-ε-caprolactone-containing thin film having a film thickness of approximately 100 nm. The spin coating conditions were that a maximum rotational number was 3,000 and a time was 30 seconds. After the spin coating, the product was allowed to stand still and dried at room temperature.

In a sample for protein adsorption test, an ultrathin film of less than 50 nm has to be formed on a metallic thin film sensor chip (measurable lower limit is 60 nm). Accordingly, poly-ε-caprolactone was dissolved in chloroform to adjust a concentration to 0.7% w/w, and the solution was spin-coated on the surface to form a poly-ε-caprolactone-containing thin film having a film thickness of approximately 51 nm. The spin coating conditions were that a maximum rotational number was 3,000 rpm and a time was 30 seconds. After the spin coating, the product was allowed to stand still and dried at room temperature.

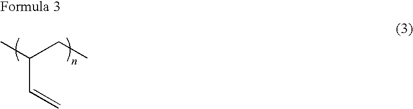

(Grafting of a Polymer Chain Having a Hydrophilic Skeleton on a Surface of a poly-ε-caprolactone)

Under the same conditions as in Examples 1 and 2, the polymer chain having a hydrophilic skeleton was grafted on the surface of the poly-ε-caprolactone. In Example 3, a copolymer of polypropylene oxide (PO) and polyethylene oxide (EO) having an amino group in one end [trade name: Jeffamine XTJ-506, molecular weight: approximately 1,000, copolymerization ratio (mol ratio) PO:EO=3:19, manufactured by HUNTSMAN], which is represented by the above formula 2, was used as a polymer chain having a hydrophilic skeleton.

### Comparative Example 1

In Comparative Example 1, a polystyrene surface was formed in the same manner as in Example 1 except that a polymer chain having a hydrophilic skeleton was not grafted.

### Comparative Example 2

In Comparative Example 2, a poly-ε-caprolactone surface was formed in the same manner as in Example 2 except that a polymer chain having a hydrophilic skeleton was not grafted.

Properties of the surfaces prepared in Examples 1 to 3 and Comparative Examples 1 and 2 were evaluated by the following (1) to (3).

(1) Contact Angle Measurement

A static contact angle to water of the surface prepared in each of Examples 1 to 3 and Comparative Examples 1 and 2 was measured using a contact angle measuring apparatus (propMaster 500, manufactured by Kaimen Kagaku). The results are shown in Table 1.

(2) Protein Adsorption

Protein adsorption on the surface prepared in each of Examples 1 to 3 and Comparative Examples 1 and 2 was evaluated with Surface Plasmon Resonance (hereinafter referred to as SPR) System (manufactured by Biacore J, Biacore) using fibronectin (purchased from SIGMA Aldrich).

A measurement principle of the apparatus is described below. When a laser beam is applied to a surface of a metallic thin film sensor chip having a thickness of 50 nm via a prism, a peculiar light absorption (decay of reflected light) is observed at a prescribed angle of a total reflection angle region. This is a phenomenon called surface plasmon resonance. When a certain molecule is immobilized on a surface of a metallic thin film sensor chip, a refractive index of an interface between the metallic thin film sensor chip surface and a liquid flowing thereon is changed to change an angle of a dark line (SPR angle) given to reflected light. The change in SPR angle induced by binding or dissociation of a molecule on the metallic thin film sensor chip surface is proportional to a change in mass of a binding molecule, and this change is recorded as a sensorgram. When a sample is added to the surface of the metallic thin film sensor chip, and molecules interact and the signal of the sensorgram is increased. When the interaction reaches equilibrium, the signal remains in a prescribed value. When the addition of the sample is finished and it is exchanged with a buffer, the binding molecules are dissociated to decrease the signal of the sensorgram. Consequently, regarding the interaction of molecules such as recognition, binding or dissociation, a complete profile can be obtained in real time. From this profile, specificity of interaction, affinity, kitics, and a concentration and an adsorption amount of a desired molecule can be found.

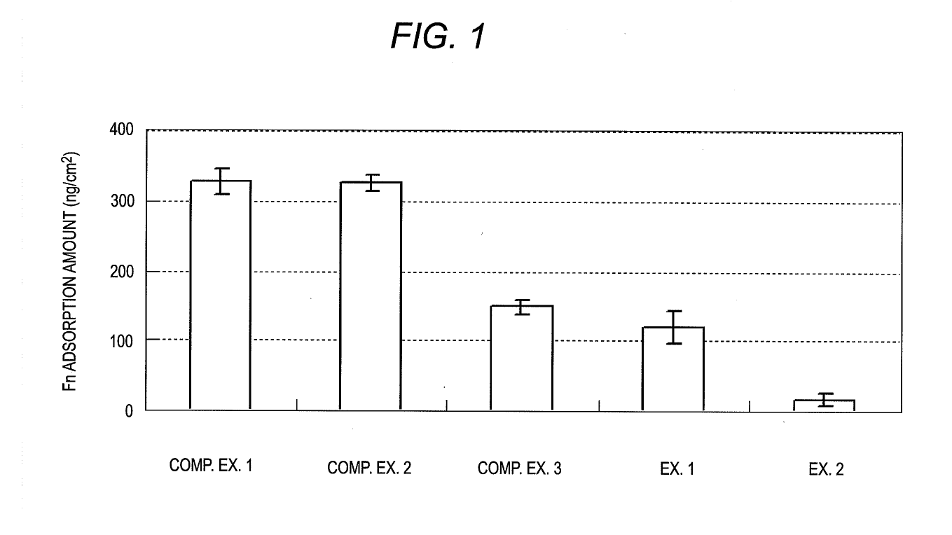

A measurement sample was set on an SPR measuring apparatus to measure an adsorption amount of fibronectin at room temperature (approximately 25° C.) with a flow rate of 30 μl of an Fn solution/min. The results are shown in Table 1 and FIG. 1.

(3) Efficiency of Cell Culture

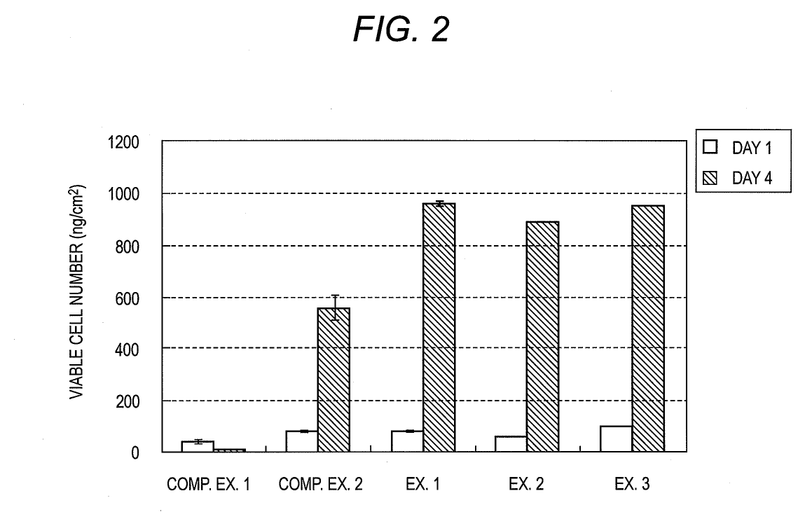

The surface prepared in each of Examples 1 to 3 and Comparative Examples 1 and 2 was sterilized with an ethylene oxide gas (40° C., 20 minutes). The sterilized surface was then inserted into a bottom of a 24-well cell culture dish (purchased from IWAKI). L6 cells (Cell Line derived from a mouse skeletal muscle: purchased from ATCC) in cell number of 40 cells/mm² were seeded, and cultured in DMEM (1% penicillin/streptomycin and 10% fetal bovine serum) at 37° C. for 4 days in the presence of 5% CO₂. As a reference, an untreated surface was also subjected to the test under the same conditions. The cell number was measured on day 1 and day 4 of the culture. The measurement was conducted using WST Assay Kit (manufactured by DOJINDO). The results are shown in Table 1 and FIG. 2.

TABLE 1

| | | Polymer having hydrophilic skeleton | | | | | Cell number (cells/mm²) | | |
|---|---|---|---|---|---|---|---|---|---|
| | | | Copolymerization | | | Fn average | Day 0 | | |
| | Hydrophobic | | ratio PO:EO (mol | Molecular | Contact | adsorption | (Seeded cell | Day 1 | Day 4 |
| | polymer | Type | ratio) | weight | angle (°) | amount (ng/cm²) | number) | (average) | (average) |
| Ex. 1 | Polystyrene | PO•EO | 3:19 | ca. 1,000 | 27.4 | 149.00 | 40 | 80.31 | 957.19 |
| | | copolymer | | | | | | | |
| Ex. 2 | Polystyrene | PO•EO | 10:31 | ca. 2,000 | 28.8 | 120.90 | 40 | 54.14 | 889.14 |
| | | copolymer | | | | | | | |
| Ex. 3 | Poly-ε- | PO•EO | 3:19 | ca. 1,000 | 56.1 | 17.35 | 40 | 102.60 | 954.63 |
| | caprolactone | copolymer | | | | | | | |
| CEx. 1 | Polystyrene | — | — | — | 99.2 | 327.10 | 40 | 40.48 | 7.54 |
| CEx. 2 | Poly-ε- | — | — | — | 77.3 | 325.40 | 40 | 84.30 | 556.36 |
| | caprolactone | | | | | | | | |

As is apparent from Table 1, the surface obtained by grafting the polymer chain having a hydrophilic skeleton on the hydrophobic polymer skeleton (Examples 1 to 3) was outstandingly hydrophilized in comparison with the surface of the hydrophobic polymer skeleton on which the polymer chain having a hydrophilic skeleton was not grafted (Comparative Examples 1 and 2).

Further, as is apparent from Table 1 and FIG. 1, the surface obtained by grafting the polymer chain having a hydrophilic skeleton on the hydrophobic polymer skeleton (Examples 1 to 3) was decreased in adhesion of the protein (Fn) in comparison with the untreated surface (Comparative Examples 1 and 2).

Moreover, as is apparent from Table 1 and FIG. 2, in Examples 1 to 3, the cell culture was conducted using the surface obtained by grafting the polymer chain having a hydrophilic skeleton on the hydrophobic polymer skeleton (Examples 1 to 3), with the result that the viable cell number on day 4 of the cell culture was outstandingly increased relative to the viable cell number on day 1 in comparison with the untreated surface (Comparative Examples 1 and 2). In addition, in Comparative Example 1, the viable cell number on day 4 of the cell culture was decreased relative to the viable cell number on day 1. This indicates that cells cannot exist on the polystyrene surface for a long period of time.

That is, it has been found that the polymer chain having a hydrophilic skeleton is grafted on the surface of the hydrophobic polymer skeleton, whereby the surface is hydrophilized to decrease the adsorption of the protein and outstandingly improve the efficiency of cell culture.

While the invention has been described in detail and with reference to specific embodiments thereof, it will be apparent to one skilled in the art that various changes and modifications can be made therein without departing from the spirit and scope thereof.

The present application is based on Japanese Patent Application No. 2007-181913 filed on Jul. 11, 2007, and the contents are incorporated herein by reference.

Further, all references cited herein are incorporated in their entireties.

### INDUSTRIAL APPLICABILITY

The cell culture substrate of the invention makes it possible to improve efficiency of the cell culture without the necessity of immobilization and adsorption of the cell adhesion substance on the substrate surface.

1. A cell culture substrate in which a polymer chain having a hydrophilic skeleton is grafted onto a surface of polystyrene or poly(ε-caprolactone) having a water contact angle of from 75° to 100°.

2. The cell culture substrate according to claim 1, wherein the surface of the polystyrene or poly(ε-caprolactone) grafted with the polymer chain having a hydrophilic skeleton has a water contact angle of from 25° to 60°.

3. The cell culture substrate according to claim 1, wherein the polymer chain having a hydrophilic skeleton is at least one member selected from the group consisting of polyethylene oxide, polypropylene oxide, and derivatives or copolymers thereof.

4. The cell culture substrate according to claim 1, wherein the polymer chain having a hydrophilic skeleton has a molecular weight of from 500 to 2,000.

5. A method for culturing cells, which comprises using the cell culture substrate according to claim 1.

6. A process for producing a cell culture substrate, comprising steps of:
(1) forming a surface of polystyrene or poly(ε-caprolactone) having a water contact angle of from 75° to 100°, and
(2) grafting a polymer chain having a hydrophilic skeleton onto the surface of the polystyrene or poly(ε-caprolactone) formed in the step (1).

7. The process according to claim 6, wherein the grafting of the polymer chain having a hydrophilic skeleton in the step (2) is conducted in accordance with plasma graft copolymerization.

8. The process according to claim 6, wherein a water contact angle of the surface of the polystyrene or poly(ε-caprolactone) grafted with the polymer chain having a hydrophilic skeleton in the step (2) is 25° to 60°.
